(19) United States
(12) Patent Application Publication
(10) Pub. No.: US 2026/0240439 A1
(43) Pub. Date: Aug. 20, 2026

(54) Sound Pickup Unit and Blood Pressure Measurement Device Comprising Same

(71) Applicant: KOROT Co., Ltd., Seoul (KR)

(72) Inventors: Ki Chul Cha, Seoul (KR); Yong Joon Yang, Yongin-si (KR)

(73) Assignee: KOROT Co., Ltd., Seoul (KR)

(21) Appl. No.: 19/161,692

(22) PCT Filed: Feb. 29, 2024
(86) PCT No.: PCT/KR2024/002625

§ 371 Date: Sep. 2, 2025

(30) Foreign Application Priority Data
Mar. 3, 2023 (KR) 10-2023-0028584
Feb. 28, 2024 (KR) 10-2024-0028749

(51) Int. Cl. A61B 5/022; A61B 5/0225

(52) U.S. Cl. CPC A61B 5/02233; A61B 5/02208; A61B 5/0225

(57) ABSTRACT

Disclosed are a sound pickup unit and a blood pressure measurement device comprising same. The disclosed blood pressure measurement device comprises: an airbag the internal pressure of which is controlled by a fluid being injected thereto or the injected fluid being discharged therefrom; a cuff which has the airbag wrapping around a part of the body of a test subject during blood pressure measurement; a sound pickup unit which is arranged to be closer to the periphery rather than the center of the cuff and collects a signal corresponding to the blood pressure of the test subject; and a control unit for measuring the blood pressure of the test subject on the basis of the signal, wherein the sound pickup unit collects the signal corresponding to the blood pressure of the test subject by being in close contact with the body of the test subject during blood pressure measurement, and the cuff is elongated in the direction in which the cuff wraps around a part of the body of the test subject.

### CROSS-REFERENCE TO RELATED APPLICATIONS

The current application is a national stage of PCT Application No. PCT/KR2024/002625 filed Feb. 29, 2024, which claims priority to Korean Patent Application No. 10-2023-0028584 filed Mar. 3, 2023 and Korean Patent Application No. 10-2024-0028749 filed Feb. 28, 2024, the disclosures of which are hereby incorporated by reference in their entireties for all purposes.

### TECHNICAL FIELD

The following description relates to a sound receiver and a blood pressure measuring device including the same.

### BACKGROUND ART

There are invasive methods and noninvasive methods of measuring blood pressure. Invasive methods are the most accurate but have issues of pain or infection, so noninvasive methods are more commonly used. Noninvasive methods may be categorized into auscultation and an oscillometric method, both of which use a cuff. Auscultation is a method of listening using a stethoscope to sound energy generated by turbulence produced during the movement of blood flow due to the opening and closing of blood vessels. The oscillometric method is primarily used in sphygmomanometers and is less accurate than auscultation.

### DISCLOSURE OF THE INVENTION

### Technical Goals

The present disclosure may collect signals (e.g., Korotkoff sound signals) corresponding to blood pressure of a subject with high accuracy when measuring blood pressure based on auscultation. The present disclosure may provide a sound receiver with enhanced accuracy in blood pressure measurement and a blood pressure measuring device including the same.

However, technical goals are not limited to the foregoing technical goals, and there may be other technical goals.

### Technical Solutions

A blood pressure measuring device according to an embodiment includes a cuff, which includes an airbag that wraps around a body part of a subject when measuring blood pressure, a sound receiver placed closer to an edge than to a center of the cuff and configured to collect signals corresponding to blood pressure of the subject, and a controller configured to measure the blood pressure of the subject based on the signals, wherein the sound receiver is configured to be in close contact with a body of the subject to collect the signals corresponding to the blood pressure of the subject and has a long shape in a direction in which the cuff wraps around the body part of the subject.

The sound receiver may include a sound collector covered by a membrane and including a groove for securing a space through which the signals transmitted to a sound collector are transmitted to a sensor, even when the membrane is pressed against the sound collector as the sound collector is pressed against the body of the subject.

The groove may be placed in the direction in which the cuff wraps around the body part of the subject.

The sound collector and the membrane may be formed from an elastic material.

The sound collector may be separable from the airbag.

The cuff may further include a D-ring at one end, a second end of the cuff may be configured to be secured to a portion of the cuff after passing through the D-ring when the cuff wraps around the body part of the subject, and the sound receiver may be placed closer to the first end than to the second end.

The blood pressure measuring device may further include an elastic body placed between the cuff and the sound receiver. The elastic body may be configured to press the sound receiver against the body of the subject in a state in which the cuff wraps around the body part of the subject.

The sound receiver may further include a second sensor configured to detect a placement angle of the sound receiver in a state in which the cuff wraps around the body part of the subject, and the controller may be configured to generate, based on the placement angle of the sound receiver detected by the second sensor, a feedback signal regarding blood pressure measurement.

The controller may be configured to generate, based on the placement angle of the sound receiver, the feedback signal for one or a combination of two or more of a posture of the subject wearing the cuff, a placement of the sound receiver in a state in which the cuff wraps around the body part of the subject, and whether a movement of the subject occurs during blood pressure measurement.

The second sensor may include one or a combination of two or more of a horizontal sensor, a gyro sensor, an acceleration sensor, and a posture detection sensor.

When measuring blood pressure, the sound receiver may be configured to transmit the signals to a sensor through an internal passage formed when internal pressure thereof is maintained greater than or equal to a predetermined threshold.

The sound receiver may be separated from the airbag so that a fluid inside may not mix with the fluid injected into the airbag.

The airbag may have an accommodation groove in which a portion of the sound receiver is accommodated.

The cuff may further include a bending plate having one end secured to the sound receiver and an other end secured to the cuff, and having a curved shape to bring the one end closer to a body than the other end.

A sound receiver for blood pressure measurement according to an embodiment includes a sound collector, which is configured to be in close contact with a body of a subject to collect signals corresponding to blood pressure of the subject and which has a long shape in a direction in which a cuff wraps around a body part of the subject, and a sensor configured to detect the signals collected by the sound collector while internal pressure of the cuff changes, wherein the sound receiver is secured to a hole placed closer to an edge than a center of the cuff, and wherein the blood pressure of the subject is measured based on the signals detected by the sensor.

The sound collector may be covered by a membrane and may include a groove for securing a space through which the signals transmitted to the sound collector are transmitted to the sensor, even when sound collector and the membrane are pressed against each other as the sound collector is pressed against the body of the subject.

The groove may be placed in the direction in which the cuff wraps around the body part of the subject.

The sound collector and the membrane may be formed from an elastic material.

The sound collector may be separable from an airbag included in the cuff, the airbag for which users may regulate internal pressure by injecting a fluid therein or discharging the injected fluid therefrom.

The cuff may further include a D-ring at a first end, while a second end of the cuff may be secured to a portion of the cuff after passing through the D-ring when the cuff wraps around the body part of the subject, and the sound collector may be placed closer to the first end than to the second end.

The sound receiver may be pressed against the body of the subject, by an elastic body placed between the cuff and the sound receiver, in a state in which the cuff wraps around the body part of the subject.

The sound receiver may further include a second sensor configured to detect a placement angle of the sound receiver in a state in which the cuff wraps around the body part of the subject, and a feedback signal regarding blood pressure measurement may be generated based on the placement angle of the sound receiver detected by the second sensor.

A blood pressure measuring device according to an embodiment includes a cuff including an airbag in which internal pressure is regulated by injecting a fluid therein or discharging the injected fluid therefrom, a sound receiver configured to collect signals corresponding to blood pressure of a subject, and a controller configured to measure the blood pressure of the subject, wherein the cuff further includes a bending plate having one end secured to the sound receiver and an other end secured to the cuff, and having a curved shape to bring the one end closer to a body of the subject than the other end.

The one end of the bending plate may be secured to a hole of the cuff, in which the sound receiver is placed.

The airbag may be positioned between the bending plate and the body of the subject.

### Effects of the Invention

According to an embodiment, a sound collector in a sound receiver may have a long shape in a direction in which a cuff wraps around a body part of a subject. The sound collector may thus collect signals corresponding to blood pressure of the subject robustly against a cuff rotation caused by a motion of wrapping the cuff around the body part of the subject.

According to an embodiment, a sound collector may, through a sound passage, effectively collect signals corresponding to blood pressure of a subject robustly against an intensity of pressing the sound collector against a body of the subject. The sound passage is a structure for securing a space through which signals transmitted to the sound collector may be transmitted to a sensor even when the sound collector and a membrane are pressed against each other as the sound collector is pressed against a body of the subject.

According to an embodiment, since signals corresponding to blood pressure of a subject may be collected most accurately when a sound receiver is positioned at a position corresponding to the brachial artery, it is necessary to minimize a rotation of a cuff that occurs when wrapping the cuff around a body part of the subject. For this purpose, the sound receiver may be placed closer to a first end at which a D-ring is placed than to a second end.

### BRIEF DESCRIPTION OF DRAWINGS

FIGS. 1 to 3 depict diagrams each illustrating a blood pressure measuring device according to an embodiment of the invention.

FIGS. 4 to 7 depict diagrams illustrating a sound receiver according to an embodiment of the invention.

FIGS. 8 to 10 depict diagrams each illustrating an example of a blood pressure measuring device according to an embodiment of the invention.

FIGS. 11 and 12 depict diagrams each illustrating an example of measuring blood pressure by a subject wearing a blood pressure measuring device, according to an embodiment of the invention.

FIGS. 13 and 14 depict diagrams illustrating a cuff including a bending plate, according to an embodiment of the invention.

FIG. 15 depict a diagram illustrating a bending plate according to an embodiment of the invention.

FIG. 16 depict a diagram illustrating a bending plate placed within a cuff, according to an embodiment of the invention.

### BEST MODE FOR CARRYING OUT THE INVENTION

The following detailed structural or functional description is provided as an example only and various alterations and modifications may be made to the embodiments. Thus, an actual form of implementation is not construed as limited to particular embodiments disclosed herein and should be understood to include all changes, equivalents, or replacements within the idea and the technical scope of the disclosure.

Terms such as first, second, and the like may be used to describe various components. These terms should be used only to distinguish one component from another component. For example, a first component may be referred to as a second component, and similarly, the second component may also be referred to as the first component.

It should be noted that when one component is described as being “connected,” “coupled,” or “joined” to another component, the first component may be directly connected, coupled, or joined to the second component, or a third component may be “connected,” “coupled,” or “joined” between the first and second components.

The singular forms “a,” “an,” and “the” are intended to include the plural forms as well, unless the context clearly indicates otherwise. It will be further understood that the terms “comprises/comprising” and/or “includes/including,” when used herein, specify the presence of stated features, integers, steps, operations, elements, and/or components, but do not preclude the presence or addition of one or more other features, integers, steps, operations, elements, components and/or groups thereof.

Unless otherwise defined, all terms used herein including technical and scientific terms have the same meanings as those commonly understood by one of ordinary skill in the art to which this disclosure pertains. Terms such as those defined in commonly used dictionaries are to be interpreted as having a meaning that is consistent with their meaning in the context of the relevant art and are not to be interpreted in an idealized or overly formal sense unless expressly so defined herein.

Hereinafter, the embodiments are described in detail with reference to the accompanying drawings. When describing the embodiments with reference to the accompanying drawings, like reference numerals refer to like components and a repeated description related thereto is omitted.

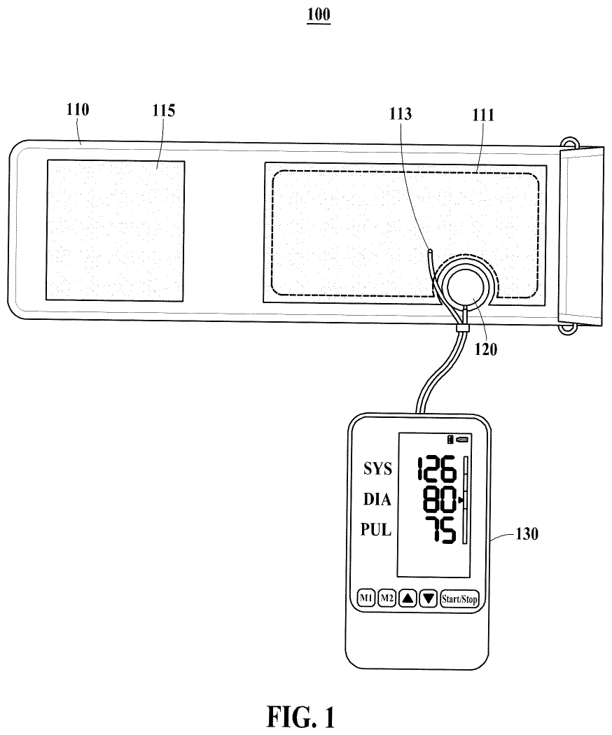

FIGS. 1 to 3 depict diagrams illustrating a blood pressure measuring device according to an embodiment of the invention.

Auscultation, in which a stethoscope is inserted under a cuff and an examiner (e.g., a doctor) directly listens to the Korotkoff sounds to measure blood pressure of the subject, is considered the most common of noninvasive methods of measuring blood pressure. The blood pressure measuring device proposed in this specification is based on auscultation, and for example, the stethoscope may be attachable to and detachable from the cuff. When measuring the blood pressure of the subject using the stethoscope (i.e., when a volume of the cuff increases and decreases due to pressurization and depressurization), the stethoscope may be pressed against a skin surface of the subject with a constant pressure. Additionally or alternatively, a hands-free structure that may be attached and detached without an assistance of a third party may be proposed.

Since the conventional method secures a stethoscope in a determined position by providing a pocket inside a cuff completely covers an entire stethoscope with the cuff, a magnitude of force that presses the stethoscope against a body of the subject changes depending on a change in internal pressure of the cuff, thereby having a negative effect on accuracy of signals detected by the stethoscope. On the contrary, the cuff proposed in this specification may ensure the stethoscope is pressed against skin of the subject with a constant pressure even when the volume of the cuff changes according to the change in the internal pressure of the cuff and may thus significantly improve the accuracy and reliability of detection of Korotkoff sound signals. In this specification, the stethoscope may also be referred to as a sound receiver.

Referring to FIG. 1, a blood pressure measuring device 100 may include but is not limited to a cuff 110, a sound receiver 120, and a controller 130.

The cuff 110 includes an airbag 111 by which internal pressure is regulated by injecting fluid therein or discharging the injected fluid therefrom, and the airbag 111 wraps around a body part of a subject when measuring blood pressure. The airbag 111 may have an accommodation groove in which a portion of the sound receiver 120 is accommodated. Referring to FIG. 1, the portion of the sound receiver 120 accommodated within the airbag 111 may be seen. When the airbag 111 does not have the accommodation groove, a width of the cuff 110 may increase by a width of the airbag 111 and a diameter of the sound receiver 120, which may make it uncomfortable for the subject to wear. When a subject whose upper arm length is insufficient wears the cuff 110 having a large width, it may be difficult for the cuff 110 to be in close contact with the body of the subject due to a structure thereof. To prevent this issue, the airbag 111 may have the accommodation groove and the sound receiver 120 may be positioned in the accommodation groove to increase space utilization of the cuff 110 and effectively reduce the width thereof. Additionally or alternatively, since a portion of the sound receiver 120 is accommodated in the accommodation groove of the airbag 111, when the subject wears the cuff 110, the sound receiver 120 may be positioned closer to a center of an upper arm than to an elbow of the subject, thereby improving adherence of the sound receiver 120 to the body of the subject. The elbow is a relatively thin area compared to the upper arm, and when the sound receiver 120 is positioned on the elbow of the subject, the adherence thereof may be reduced.

For example, the body part of the subject may represent the upper arm but is not limited to the example described above.

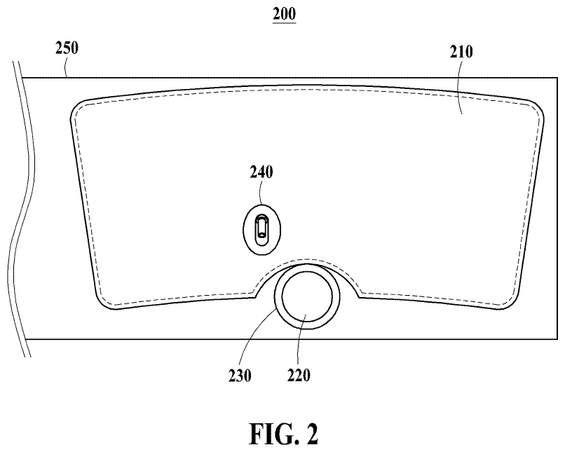

Referring to FIG. 2, a cuff 200 may include an airbag 210, a hole 220, a support 230, a fluid gate 240, and a cuff sheet 250.

The cuff sheet 250 may include the airbag 210 therein and may have a belt shape so that the airbag 210 may wrap around a body part of a subject when measuring blood pressure. The cuff sheet 250 may also be referred to as a cuff cloth or band. The cuff sheet 250 includes the hole 220 therein.

The airbag 210 may be a part of how internal pressure is regulated by injecting a fluid therein or discharging the injected fluid therefrom. For example, when a fluid such as air is injected into the airbag 210, the internal pressure may increase. On the contrary, when a fluid injected into the airbag 210 is discharged, the internal pressure may decrease. The fluid gate 240 may be a part through which a fluid is injected into the airbag 210 or through which the previously injected fluid is discharged. Although not shown in FIG. 2, the fluid gate 240 may be connected to a separate flow path to receive or discharge a fluid.

The hole 220 is a portion to which a sound receiver (e.g., the sound receiver 120 of FIG. 1) is secured, and may be placed closer to an edge of the cuff sheet 250 than to a center of the cuff sheet 250. In other words, the hole 220 may be placed on a side portion of the cuff sheet 250 and secure the sound receiver. Although not shown in FIG. 2, the sound receiver may be attached to or detached from the hole 220. The hole 220 may have a shape corresponding to the sound receiver to secure the sound receiver, and may have a ring shape in the example of FIG. 2. Any other shape that is capable of securing the sound receiver may be adopted without limitation. The hole 220 may be placed according to the accommodation groove of the airbag 111 described with reference to FIG. 1. The sound pickup device may be a device that comes into contact with a body of the subject and detects Korotkoff sound signals.

The support portion 230 is a part that supports the sound receiver secured to the hole 220 and may be made of, for example, a harder material than the airbag 210. The support portion 230 may completely surround the hole 220 and may have a ring shape in the example of FIG. 2. Compared to the structure described above, in which the entire sound receiver is completely covered by the cuff 200 and the magnitude of the force that presses the stethoscope against the body of the subject changes significantly depending on a change in internal pressure of the cuff 200, the structure of FIG. 2 may effectively reduce an influence of a change in force pressing the body part of the subject caused by the change in the internal pressure of the cuff 200 on force applied to the sound receiver that is pressed against the body of the subject.

Referring again to FIG. 1, the cuff 110 may further include a fluid gate 113 and a Velcro 115. The fluid gate 113 may be a part through which a fluid is injected into the airbag 111 inside or through which the previously injected fluid is discharged. The Velcro 115 may be used to secure the cuff 110 around a body part of a subject.

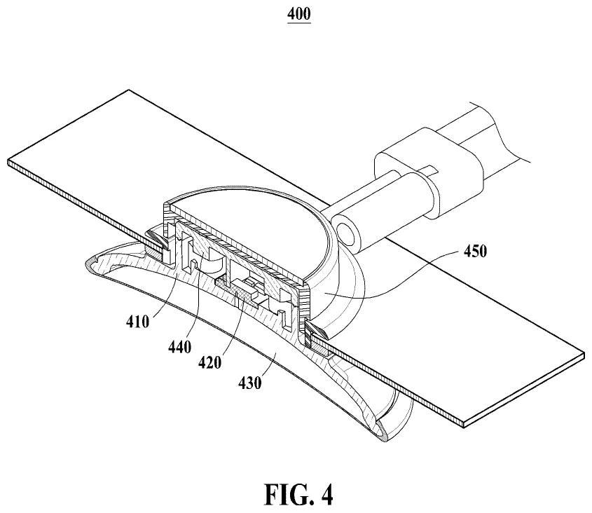

The sound receiver 120 may be in close contact with a body of the subject in a state in which the cuff 110 wraps around the body part of the subject and may be used to detect signals (e.g., Korotkoff sound signals, etc.) corresponding to blood pressure of the subject. The receiver 120 may be secured to a hole placed closer to an edge than to a center of the cuff 110. The sound receiver 120 may include a sound collector, which is in close contact with the body of the subject when measuring blood pressure to collect the signals corresponding to the blood pressure of the subject and has a long shape in a direction in which the cuff 110 wraps around the body part of the subject, and a sensor that detects the signals collected by the sound collector. The sound receiver 120 is described in detail with reference to FIGS. 4 to 7.

According to an embodiment, an elastic body (not shown in the drawing) (e.g., a spring, etc.) may be placed between the cuff 110 and the sound receiver 120 to press the sound receiver 120 against the body of the subject in a state in which the cuff 110 wraps around the body part of the subject. It may be expected that the sound receiver 120 in the cuff 110 wrapping around the body part of the subject is pressed closer, by the elastic body, against the body of the subject. An effect may be expected which is similar to when the examiner presses the stethoscope against the body of the subject with a hand. By pressing the sound receiver 120 more strongly against the body of the subject through the elastic body, the sound receiver 120 may be sealed by the body of the subject and may thus effectively detect the signals corresponding to the blood pressure of the subject when measuring blood pressure. Additionally or alternatively, the elastic body may act as a buffer between the cuff 110 and the subject.

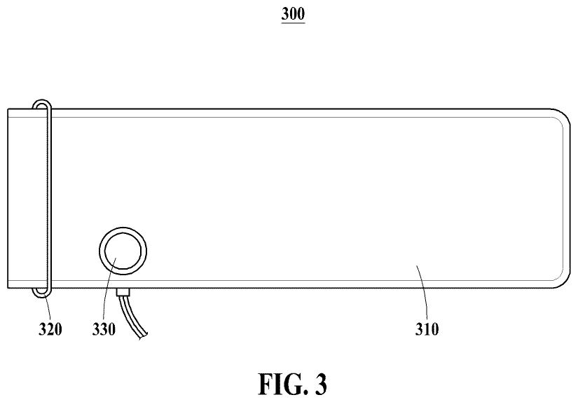

A cuff 310 and a sound receiver 330 of a blood pressure measuring device 300 are further described with reference to FIG. 3. FIG. 3 may illustrate the cuff 110 and the sound receiver 120 of FIG. 1 turned over. The cuff 310 may include a D-ring 320 at a first end. When the cuff 310 wraps around a body part of a subject, a second end of the cuff 310 may be secured to a portion of the cuff 310 after passing through the D-ring 320. Here, the second end may be an end different from the first end of the cuff 310.

The sound receiver 330 may be placed closer to the first end, at which the D-ring 320 may be placed, than to the second end. By this placement structure of the sound receiver 330, it may be possible to minimize a change in a position of the sound receiver 330 caused by a motion of the subject or the examiner wrapping the cuff 310 around the body part of the subject. For example, the subject or the examiner may pass the second end of the cuff 310 through the D-ring 320 and pull the second end of the cuff 310 so that the cuff 310 may be in close contact with the body part of the subject, and subsequently may secure the second end of the cuff 310 to a portion of the cuff 310 using a Velcro. While the second end of the cuff 310 is passed through the D-ring 320 and pulled, when the cuff 310 rotates in a state of wrapping around the body part of the subject, the position of the sound receiver 330 may also rotate. Since signals corresponding to blood pressure of the subject may be collected most accurately when the sound receiver 330 is positioned at a position corresponding to the brachial artery, it is necessary to minimize a rotation of the cuff 310 that occurs when wrapping the cuff 310 around the body part of the subject, and for this purpose, the sound receiver 330 may be placed closer to the first end at which the D-ring 320 is placed than to the second end.

Referring again to FIG. 1, the controller 130 measures the blood pressure of the subject based on the detected signals. For example, the controller 130 may display systolic blood pressure and diastolic blood pressure of the measured blood pressure of the subject on a display.

FIGS. 4 to 7 depict diagrams illustrating a sound receiver according to an embodiment of the invention.

Referring to FIG. 4, a cross-sectional view of the sound receiver 400 in a first axis direction is illustrated. The sound receiver 400 may include a sound collector 410, a sound sensor 420, and a membrane 430. Additionally or alternatively, the sound receiver 400 may further include an upper cover 440 and a decorative cover 450.

The sound collector 410 is in close contact with a body of a subject when measuring blood pressure to collect signals corresponding to blood pressure of the subject and has a long shape in a direction in which a cuff wraps around a body part of the subject. In the example of FIG. 4, the cross-sectional direction may match the direction in which the cuff wraps around the body part of the subject, and the sound collector 410 may also have a long shape in that direction. Since the sound collector 410 has a long shape in that direction, even when the cuff rotates due to a motion of wrapping the cuff around the body part of the subject, the brachial artery may be effectively positioned within a collection range of the sound collector 410. In other words, the sound collector 410 has a long shape in the direction in which the cuff wraps around the body part of the subject and may thus collect the signals corresponding to the blood pressure of the subject robustly against the cuff rotation caused by the motion of wrapping the cuff around the body part of the subject.

The sound collector 410 may be covered by the membrane 430. The membrane 430 may include a diaphragm. The diaphragm may be made of a variety of materials, including polyvinyl chloride (PVC), for optimal sound collection. The sound collector 410 and the membrane 430 may be formed from an elastic material to naturally bend and adhere to each other when the cuff is fastened. The sound collector 410 may be separated from an airbag of the cuff.

The sound sensor 420 may detect signals collected by the sound collector 410. For example, the signals collected by the sound collector 410 may be Korotkoff sound signals.

The upper cover 440 may connect and secure the sound collector 410 and the sound sensor 420 to each other to prevent sound from leaking. The decorative cover 450 may cover the receiver 400 and a cable and provide design aesthetics. According to an embodiment, the decorative cover 450 may include a light emitting diode (LED) that indicates an operation of the cuff and the like.

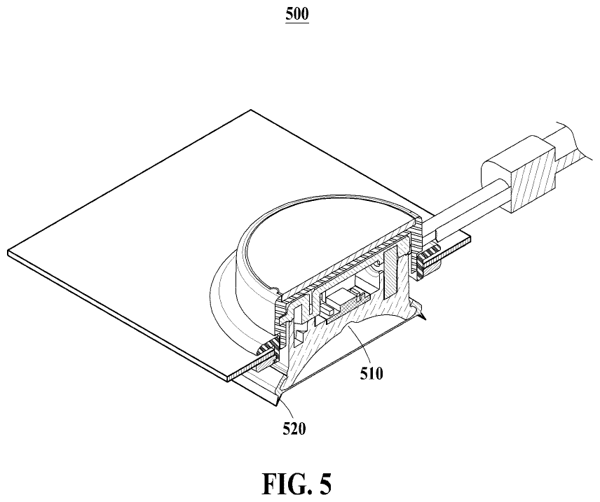

A sound collector is described with reference to FIG. 5. FIG. 5 illustrates an example of a cross-sectional view in a second axis direction. Here, the second axis direction may be perpendicular to the first axis direction of FIG. 4.

The sound collector may include a sound passage 510 that is placed in a direction in which a cuff wraps around a body part of a subject. The sound passage 510 may be a groove that is partially recessed inward in a shape of the sound collector. The sound passage 510 may be a structure for securing a space through which signals transmitted to the sound collector may be transmitted to a sensor even when the sound collector and a membrane are pressed against each other as the sound collector is pressed against the body of the subject. Through the sound passage 510, the sound collector may effectively collect signals corresponding to blood pressure of the subject robustly against an intensity of pressing the sound collector against the body of the subject.

In an embodiment, a sealing protrusion 520 may be placed on an edge of the sound collector. The sealing protrusion 520 may seal the sound collector to the body of the subject when the sound receiver 500 comes into contact with the body of the subject, so that the signal corresponding to the blood pressure of the subject may be transmitted well to the sound collector.

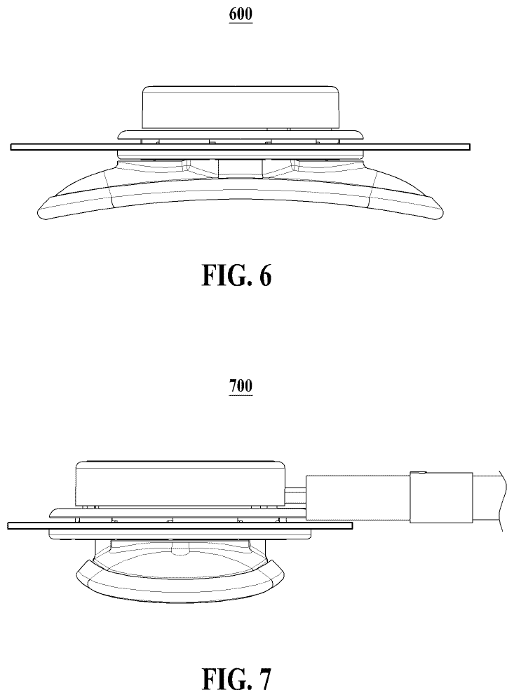

Referring to FIG. 6, a lateral view of the sound receiver 600 in the first axis direction is illustrated. Referring to FIG. 7, a lateral view of a sound receiver 700 in the second axis direction is illustrated.

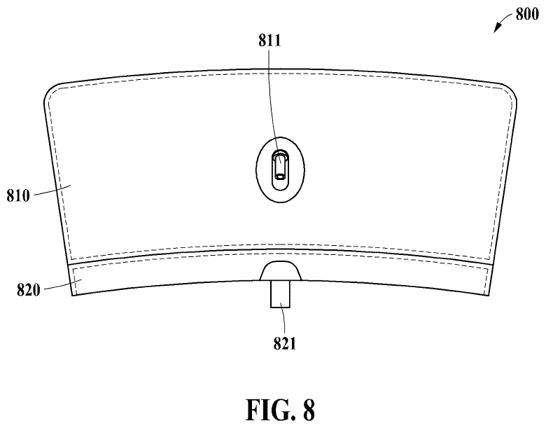

FIGS. 8 to 10 depict diagrams each illustrating an example of a blood pressure measuring device according to an embodiment of the invention.

Referring to FIG. 8, a blood pressure measuring device 800 may include a cuff (not shown in the drawing), a sound receiver 820, and a controller (not shown in the drawing).

The cuff may include an airbag 810 by which internal pressure is regulated by injecting fluid therein or discharging the injected fluid therefrom, and the airbag 810 may wrap around a body part of a subject when measuring blood pressure. For example, the body part of the subject may represent the upper arm but is not limited to the example described above. Through a first fluid gate 811 formed in the airbag 810, the fluid may be injected into the airbag 810 or the previously injected fluid may be discharged. Although not shown in FIG. 8, the first fluid gate 811 may be connected to a separate flow path to receive or discharge a fluid.

The sound receiver 820 may be in close contact with a body of the subject in a state in which the cuff wraps around the body part of the subject and collect signals (e.g., Korotkoff sound signals, etc.) corresponding to blood pressure of the subject. The sound receiver 820 may be placed adjacent to an edge of the cuff or the airbag 810. The receiver 820 may have a long shape in a direction in which the cuff wraps around the body part of the subject and may be in close contact with the body of the subject when measuring blood pressure to collect the signals corresponding to the blood pressure of the subject. When measuring blood pressure, the sound receiver 820 may transmit the signals to a sensor through an internal passage formed when internal pressure thereof is maintained greater than or equal to a predetermined threshold. Before measuring blood pressure, a fluid may be injected into the sound receiver 820 so that the internal pressure may be maintained greater than or equal to the predetermined threshold during blood pressure measurement. For example, since the sound receiver 820 has a long shape in a direction of wrapping around the body part of the subject, when the airbag 810 wraps around the body part of the subject to measure blood pressure, the sound receiver 820 may also wrap around the body part of the subject. In this case, the internal pressure of the sound receiver 820 may be maintained greater than or equal to the predetermined threshold when measuring blood pressure in order to prevent the sound receiver 820 from being partially folded and blocking the internal passage.

Since the sound receiver 820 has a long shape in the direction of wrapping around the body part of the subject, even when the cuff rotates due to a motion of wrapping the cuff around the body part of the subject, the brachial artery may be effectively positioned within a sound receiving range of the sound receiver 820. In other words, the sound receiver 820 has a long shape in the direction in which the cuff wraps around the body part of the subject and may thus collect the signals corresponding to the blood pressure of the subject robustly against the cuff rotation caused by the motion of wrapping the cuff around the body part of the subject.

The sound receiver 820 may be separated from the airbag 810 so that the fluid inside may not mix with the fluid injected into the airbag 810. While internal pressure of the airbag 810 changes when measuring blood pressure, the internal pressure of the sound receiver 820 needs to be maintained greater than or equal to a determined level, so the sound receiver 820 may be separated from the airbag 810 so that the fluids inside do not mix with each other. In other words, an inner pocket of the sound receiver 820 and an inner pocket of the airbag 810 are not connected to each other.

Through a second fluid gate 821 formed in the sound receiver 820, the fluid may be injected into the sound receiver 820 or the previously injected fluid may be discharged. Although not shown in FIG. 8, the second fluid gate 821 may be connected to a separate flow path to receive or discharge a fluid.

According to an embodiment, a sensor (not shown in the drawing) that detects the signals collected by the sound receiver 820 may be located inside the sound receiver 820. For example, the sensor may be located at a center rather than at either end of the sound receiver 820 to detect the signals (e.g., Korotkoff sound signals) collected by the sound receiver 820 and transmit information on the detected signals to the controller. The position of the sensor is not limited to the examples described above.

Since the description provided above applies as is to the cuff, the sensor, and the controller, a more detailed description thereof is omitted.

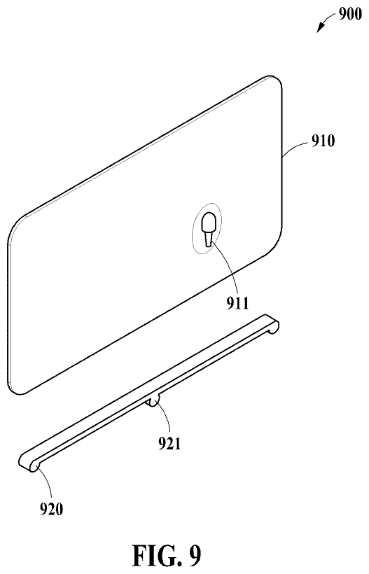

Referring to FIG. 9, a blood pressure measuring device 900 may include a cuff (not shown in the drawing), a sound receiver 920, and a controller (not shown in the drawing). The airbag 810 and the sound receiver 820 may be attached to each other and formed as an integral body as shown in FIG. 8. Alternatively, an airbag 910 and the sound receiver 920 may be formed as a separate type, without being attached to each other, as shown in FIG. 9. A first fluid gate 911 may be a part through which a fluid is injected into the airbag 810 or through which the previously injected fluid is discharged. A second fluid gate 921 may be a part through which a fluid is injected into the sound receiver 920 or through which the previously injected fluid is discharged. Since the description provided above with reference to FIG. 8 applies as is to the blood pressure measuring device 900, a more detailed description thereof is omitted.

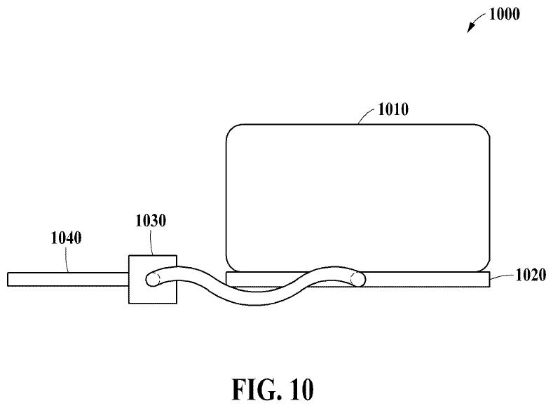

Referring to FIG. 10, a blood pressure measuring device 1000 may include a cuff (not shown in the drawing), a sound receiver 1020, a connector 1030, and a controller 1040. The cuff may include an airbag 1010 by which internal pressure is regulated by injecting fluid therein or by discharging the injected fluid therefrom. The connector 1030 may be connected to a sensor in the sound receiver 1020, and the controller 1040 may measure blood pressure of a subject based on signals from the sensor connected via the connector 1030. Since the description provided above with reference to FIGS. 8 and 9 applies as is to the blood pressure measuring device 1000, a more detailed description thereof is omitted.

The sound receivers 820, 920, and 1020 described with reference to FIGS. 8 to 10 are for collecting signals and may also be referred to as a microphone bag.

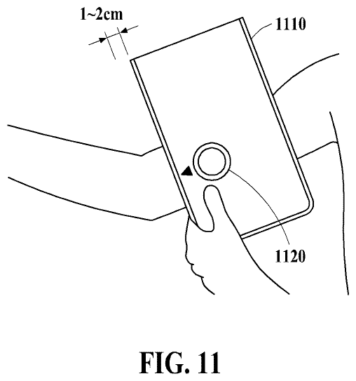

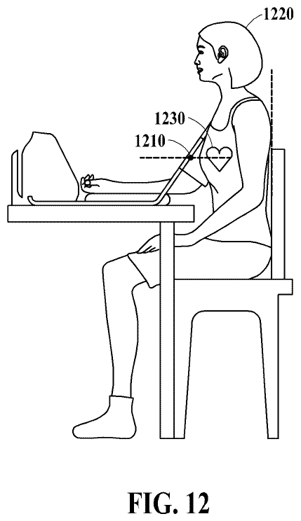

FIG. 11 and FIG. 12 depict diagrams each illustrating an example of measuring blood pressure by a subject wearing a blood pressure measuring device, according to an embodiment of the invention.

Referring to FIG. 11, the subject may wear the blood pressure measuring device on an upper arm. The subject may wear the blood pressure measuring device so that a lower end of a cuff 1110 may be approximately 1-2 cm above an elbow crease. The receiver/cuff 1110 may be placed on a position corresponding to the brachial artery.

According to an embodiment, the sound receiver 1120 may further include a second sensor that detects a placement angle of the sound receiver 1120 in a state in which the cuff 1110 wraps around a body part of the subject. As the sound receiver 1120 is coupled to a hole of the cuff 1110, the placement angle of the sound receiver 1120 may vary depending on a posture of the subject wearing the cuff 1110 for blood pressure measurement or a state of the cuff 1110 wrapping around the body part of the subject. Additionally or alternatively, the placement angle of the sound receiver 1120 may change depending on a movement of the subject that may occur during blood pressure measurement. Using these characteristics, a controller may generate a feedback signal related to blood pressure measurement, based on the placement angle of the sound receiver 1120 detected by the second sensor.

For example, the controller may generate, based on the placement angle of the sound receiver 1120, a feedback signal for one or a combination of two or more of a posture of the subject wearing the cuff 1110, the placement of the sound receiver 1120 in a state in which the cuff 1110 wraps around the body part of the subject, and whether a movement of the subject occurs during blood pressure measurement.

Referring to FIG. 12, in order to achieve high accuracy in blood pressure measurement, a cuff 1210 wrapped around a body part of a subject 1220 needs to be positioned at a same height as a heart 1230 of the subject 1220, and an appropriate posture of the subject 1220 for blood pressure measurement may thus be required. When a value corresponding to an angle of a sound receiver that occurs naturally in the appropriate posture is not detected by a second sensor, a controller may generate a feedback signal indicating that a measurement posture of the subject 1220 is not appropriate. Additionally or alternatively, in order to accurately determine a change in blood pressure of the subject 1220 over time, it may be important to take a same posture each time blood pressure is measured. When the controller determines that a placement angle of the sound receiver detected by the second sensor is different from that detected in a previous blood pressure measurement, the controller may generate a feedback signal indicating that the measurement posture of the subject 1220 is different from before.

Since signals corresponding to the blood pressure of the subject 1220 are collected from the brachial artery of the subject 1220, the sound receiver may need to be placed at a position corresponding to the brachial artery to more accurately collect the signals corresponding to the blood pressure of the subject 1220. When the placement angle of the sound receiver detected by the second sensor does not correspond to an angle formed when the sound receiver is placed at a position corresponding to the brachial artery due to a rotation of the cuff 1210 wrapping around the body part of the subject 1220, the controller may generate a feedback signal indicating that the placement of the sound receiver in a state in which the cuff 1210 wraps around the body part of the subject 1220 is not appropriate.

While measuring blood pressure, the subject 1220 may need to remain motionless and take a stable posture. Accordingly, when the angle of the placement of the sound receiver detected by the second sensor changes by a determined level or more during blood pressure measurement, the controller may generate a feedback signal indicating that a movement of the subject 1220 has occurred during blood pressure measurement and/or a feedback signal requesting not to move during blood pressure measurement.

The generated feedback signal may be provided to the subject 1220 or an examiner in various forms. For example, the feedback signal may take various forms, such as a voice message, a visual message, a warning light, and the like, and is not limited to the examples described above.

The second sensor may include one or more of a horizontal sensor, a gyro sensor, an acceleration sensor, and a posture detection sensor.

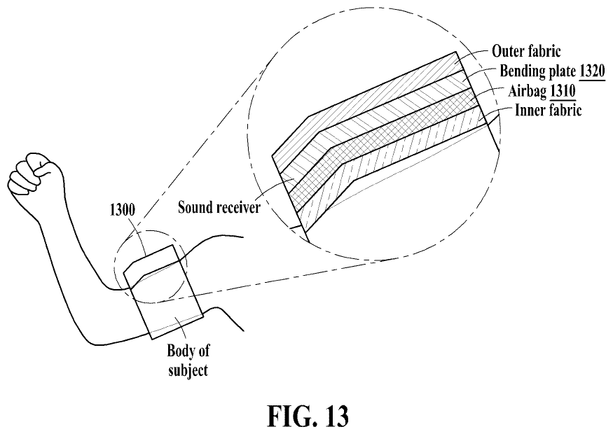

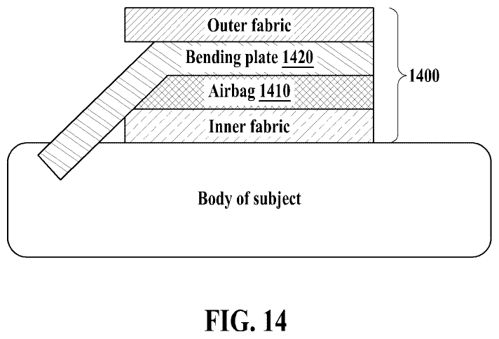

FIGS. 13 and 14 depict diagrams illustrating a cuff including a bending plate, according to an embodiment of the invention.

Referring to FIG. 13, an example is shown in which a cuff 1300 wraps around a body part (e.g., an upper arm) of a subject. The cuff 1300 may include an airbag 1310 and a bending plate 1320.

The body part of the subject may not have constant thickness. For example, the upper arm may have a cone shape that tapers from a shoulder to an elbow. Due to this body structure, the cuff 1300 may not be in close enough contact with a body of the subject, and accordingly, a detection intensity of signals (e.g., Korotkoff sounds, etc.) corresponding to blood pressure may not be sufficiently high. In order to collect signals with high accuracy, the subject or an examiner (e.g., medical staff) may press a sound receiver so that the sound receiver may be in close contact with the body of the subject. However, it may not be easy to maintain a constant pressure on the sound receiver while measuring blood pressure, and an act of pressing itself may be uncomfortable. The bending plate 1320 has one end secured to the sound receiver and an other end secured to the cuff 1300, and may have a curved shape so that the one end may be closer to the body than the other end to ensure that the sound receiver is in close contact with the body of the subject at a constant strength while measuring blood pressure, thereby enabling the signals corresponding to the blood pressure to be obtained with high accuracy. In the bending plate 1320 shown in FIG. 13, the bent portion on the left may be the one end secured to the sound receiver, and the flat portion on the right may be the other end secured to the cuff 1300.

Referring to FIG. 14, a cross-sectional view of a cuff 1400 including an airbag 1410 and a bending plate 1420 is illustrated. The airbag 1410 may be positioned between the bending plate 1420 and a body of a subject, so that the airbag 1410 may be pressed closer against the body of the subject by the bending plate 1420, thereby preventing pressurization of the airbag 1410 from being relieved.

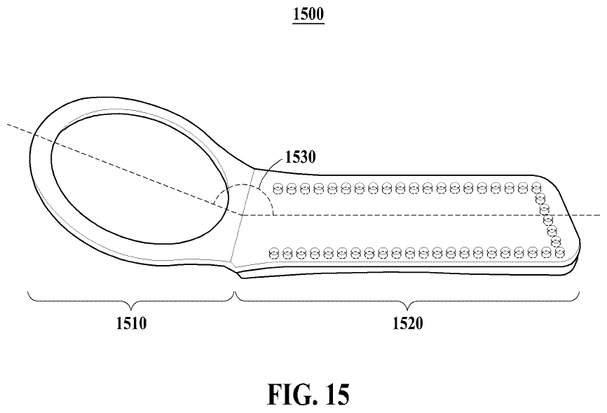

FIG. 15 depict a diagram illustrating a bending plate according to an embodiment of the invention.

Referring to FIG. 15, a bending plate 1500 may be divided into one end 1510 and an other end 1520. In FIG. 15, the one end 1510 and the other end 1520 are illustrated as having a certain angle 1530, but this is only an example of a structure for bringing the one end 1510 closer to a body of a subject than the other end 1520. The structure is not limited to the above-described example, and any structure (e.g., a curved shape, etc.) for bringing the one end 1510 closer to the body of the subject than the other end 1520 may be included without limitation.

The one end 1510 may include a hole secured to a sound receiver, and the hole of the one end 1510 may correspond to a hole of a cuff. The other end 1520 may be secured to the cuff.

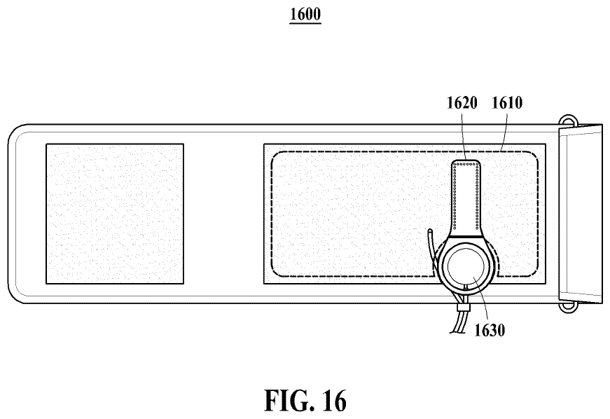

FIG. 16 depict a diagram illustrating a bending plate placed within a cuff, according to an embodiment of the invention.

Referring to FIG. 16, a cuff 1600 may include an airbag 1610 and a bending plate 1620. A sound receiver 1630 may be secured to a hole placed at one end of the bending plate 1620. An other end of the bending plate 1620 may be placed toward a shoulder of a subject. The bending plate 1620 may be placed in a length direction of an arm so that the subject does not feel uncomfortable even when the cuff 1600 wraps around a body part of the subject. Here, the length direction of the arm may refer to a direction perpendicular to a direction in which the cuff 1600 wraps around a body part of the subject.

The embodiments described herein may be implemented using a hardware component, a software component, and/or a combination thereof. For example, a processing device may be implemented using one or more general-purpose or special-purpose computers, such as, for example, a processor, a controller, an arithmetic logic unit (ALU), a digital signal processor (DSP), a microcomputer, an FPGA, a programmable logic unit (PLU), a microprocessor, or any other device capable of responding to and executing instructions in a defined manner. The processing device may run an operating system (OS) and one or more software applications that run on the OS. The processing device may also access, store, manipulate, process, and create data in response to execution of the software. For purpose of simplicity, the processing device is described as singular. However, one of ordinary skill in the art will appreciate that a processing device may include multiple processing elements and/or multiple types of processing elements. For example, the processing device may include a plurality of processors, or a single processor and a single controller. Additionally or alternatively, a different processing configuration is possible, such as one including parallel processors.

The software may include a computer program, a piece of code, instructions, or one or more combinations thereof, to independently or collectively instruct or configure the processing device to operate as desired. Software and/or data may be embodied permanently or temporarily in any type of machine, component, physical or virtual equipment, computer storage medium or device, or in a propagated signal wave for the purpose of being interpreted by the processing device or providing instructions or data to the processing device. The software may also be distributed over network-coupled computer systems so that the software is stored and executed in a distributed fashion. The software and data may be stored in a non-transitory computer-readable recording medium.

The methods according to the embodiments may be recorded in non-transitory computer-readable media including program instructions to implement various operations of the embodiments. The media may also include the program instructions, data files, data structures, and the like alone or in combination. The program instructions recorded on the media may be those specially designed and constructed for the embodiments, or they may be of the kind well-known and available to those having skill in the computer software arts. Examples of non-transitory computer-readable media include magnetic media such as hard disks, floppy disks, and magnetic tape; optical media such as compact disc read-only memory (CD-ROM) discs and digital video discs (DVDs); magneto-optical media such as floptical disks; and hardware devices that are specially configured to store and perform program instructions, such as read-only memory (ROM), random-access memory (RAM), flash memory, and the like. Examples of program instructions include both machine code, such as those produced by a compiler, and files containing high-level code that may be executed by the computer using an interpreter. The above-described hardware devices may be configured to act as one or more software modules in order to perform the operations of the above-described embodiments, or vice versa.

Although the embodiments have been described with reference to the limited number of drawings, one of ordinary skill in the art may apply various technical modifications and variations based thereon. For example, suitable results may be achieved if the described techniques are performed in a different order and/or if components in a described system, architecture, device, or circuit are combined in a different manner and/or replaced or substituted by other components or their equivalents.

Therefore, other implementations, other embodiments, and equivalents to the claims are also within the scope of the following claims.

1. A blood pressure measuring device comprising:
a cuff comprising an airbag of which internal pressure is regulated by injecting a fluid therein or discharging the injected fluid therefrom, wherein the airbag wraps around a body part of a subject when measuring blood pressure;
a sound receiver placed closer to an edge than to a center of the cuff and configured to collect signals corresponding to blood pressure of the subject; and
a controller configured to measure the blood pressure of the subject based on the signals,
wherein the sound receiver is configured to be in close contact with a body of the subject to collect the signals corresponding to the blood pressure of the subject and has a long shape in a direction in which the cuff wraps around the body part of the subject.

2. The blood pressure measuring device of claim 1, wherein the sound receiver comprises: a sound collector covered by a membrane and comprising a groove for securing a space through which the signals transmitted to a sound collector are transmitted to a sensor, even when the membrane is pressed against the sound collector as the sound collector is pressed against the body of the subject.

3. The blood pressure measuring device of claim 2, wherein the groove is placed in the direction in which the cuff wraps around the body part of the subject.

4. The blood pressure measuring device of claim 2, wherein the sound collector and the membrane are formed of an elastic material.

5. The blood pressure measuring device of claim 2, wherein the sound collector is separable from the airbag.

6. The blood pressure measuring device of claim 1,
wherein the cuff further comprises a D-ring at one end;
wherein a second end of the cuff is configured to be secured to a portion of the cuff after passing through the D-ring when the cuff wraps around the body part of the subject; and
wherein the sound receiver is placed closer to the first end than to the second end.

7. The blood pressure measuring device of claim 1, further comprising: an elastic body placed between the cuff and the sound receiver and configured to press the sound receiver against the body of the subject in a state in which the cuff wraps around the body part of the subject.

8. The blood pressure measuring device of claim 1,
wherein the sound receiver further comprises a second sensor configured to detect a placement angle of the sound receiver in a state in which the cuff wraps around the body part of the subject, and
wherein the controller is configured to generate, based on the placement angle of the sound receiver detected by the second sensor, a feedback signal regarding blood pressure measurement.

9. The blood pressure measuring device of claim 8, wherein the controller is configured to generate, based on the placement angle of the sound receiver, the feedback signal for one or a combination of two or more of:
a posture of the subject wearing the cuff;
a placement of the sound receiver in a state in which the cuff wraps around the body part of the subject; and
whether a movement of the subject occurs during blood pressure measurement.

10. The blood pressure measuring device of claim 8, wherein the second sensor comprises one or a combination of two or more of: a horizontal sensor, a gyro sensor, an acceleration sensor, and a posture detection sensor.

11. The blood pressure measuring device of claim 1, wherein, when measuring blood pressure, the sound receiver is configured to: transmit the signals to a sensor through an internal passage formed when internal pressure thereof is maintained greater than or equal to a predetermined threshold.

12. The blood pressure measuring device of claim 1, wherein the sound receiver is separated from the airbag so that a fluid inside the sound receiver does not mix with the fluid injected into the airbag.

13. The blood pressure measuring device of claim 1, wherein the airbag has an accommodation groove in which a portion of the sound receiver is accommodated.

14. The blood pressure measuring device of claim 1, wherein the cuff further comprises: a bending plate having one end secured to the sound receiver and an other end secured to the cuff, and having a curved shape to bring the one end closer to a body than the other end.

15. A sound receiver for blood pressure measurement, the sound receiver comprising:
a sound collector, which is configured to be in close contact with a body of a subject to collect signals corresponding to blood pressure of the subject and which has a long shape in a direction in which a cuff wraps around a body part of the subject; and
a sensor configured to detect the signals collected by the sound collector while internal pressure of the cuff changes,
wherein the sound receiver is secured to a hole placed closer to an edge than a center of the cuff, and
wherein the blood pressure of the subject is measured based on the signals detected by the sensor.

16. The sound receiver of claim 15, wherein the sound collector is covered by a membrane, and the sound collector comprises a groove for securing a space through which the signals transmitted to the sound collector are transmitted to the sensor, even when sound collector and the membrane are pressed against each other as the sound collector is pressed against the body of the subject.

17. The sound receiver of claim 16, wherein the groove is placed in the direction in which the cuff wraps around the body part of the subject.

18-22. (canceled)

23. A blood pressure measuring device comprising:
a cuff comprising an airbag of which internal pressure is regulated by injecting a fluid therein or discharging the injected fluid therefrom;
a sound receiver configured to collect signals corresponding to blood pressure of a subject; and
a controller configured to measure the blood pressure of the subject,
wherein the cuff further comprises: a bending plate having one end secured to the sound receiver and an other end secured to the cuff, and having a curved shape to bring the one end closer to a body of the subject than the other end.

24. The blood pressure measuring device of claim 23, wherein the one end of the bending plate is secured to a hole of the cuff, in which the sound receiver is placed.

25. The blood pressure measuring device of claim 23, wherein the airbag is positioned between the bending plate and the body of the subject.
